(19) United States
(12) Patent Application Publication
(10) Pub. No.: US 2022/0284313 A1
(43) Pub. Date: Sep. 8, 2022

(54) LEARNING DEVICE, PREDICTION DEVICE, LEARNING METHOD, PREDICTION METHOD, AND PROGRAM

(71) Applicant: NIPPON TELEGRAPH AND TELEPHONE CORPORATION, Tokyo (JP)

(72) Inventors: Maya OKAWA, Tokyo (JP); Tomoharu IWATA, Tokyo (JP); Hiroyuki TODA, Tokyo (JP); Takeshi KURASHIMA, Tokyo (JP); Yusuke TANAKA, Tokyo (JP)

(73) Assignee: NIPPON TELEGRAPH AND TELEPHONE CORPORATION, Tokyo (JP)

(21) Appl. No.: 17/624,564

(22) PCT Filed: Jul. 4, 2019
(86) PCT No.: PCT/JP2019/026700

§ 371 Date: Jan. 3, 2022

(51) Int. Cl. G06N 5/02; G06N 5/04

(52) U.S. Cl. CPC G06N 5/022; G06N 5/04

(57) ABSTRACT

A learning device includes a learning unit that learns parameters for determining an occurrence probability of an event at each time and each location on the basis of history information relating to the event, the history information including a time, a location, and an event type, and features of an area corresponding to the location, so that a likelihood expressing a combined effect of the event type and the features of the area on the event is optimized.

### TECHNICAL FIELD

The technology in the disclosure relates to a learning device, a prediction device, a learning method, a prediction method, and a program.

### BACKGROUND ART

Techniques for predicting an event are available in the prior art. For example, to predict an event, event data are expressed as a series of events and described using a model known as a point process. A spatio-temporal point process is widely used to model events that are spread out in space-time. For example, a self-exciting spatio-temporal point process known as the Hawkes process is widely used to model earthquakes or conflicts (see NPL 1 and NPL 2).

### CITATION LIST

### Non Patent Literature

[NPL 1] Reinhart, A. (2018). A review of self-exciting spatio-temporal point processes and their applications. Statistical Science, 3 3(3), 299-318.
[NPL 2] Louie, K., Masaki, M., Allenby, M. (2010). A point process model for simulating gang-on-gang violence.

### SUMMARY OF THE INVENTION

### Technical Problem

With existing methods, however, the effects of external factors relating to each event on the occurrence probability of the event cannot be sufficiently reflected, and therefore the prediction precision cannot be said to be sufficient.

An object of the present disclosure is to provide a learning device, a prediction device, a learning method, a prediction method, and a program for ascertaining features of an area in order to predict the occurrence of an event with a high degree of precision.

### Means for Solving the Problem

A first aspect of the present disclosure is a learning device including a learning unit that learns parameters for determining an occurrence probability of an event at each time and each location on the basis of history information relating to the event, the history information including a time, a location, and an event type, and features of an area corresponding to the location, so that a likelihood expressing a combined effect of the event type and the features of the area on the event is optimized.

A second aspect of the present disclosure is a prediction device including a search unit that receives a predicted time and a predicted location, and a prediction unit that predicts the occurrence of an event at the predicted time and the predicted location on the basis of pre-learned parameters for determining an occurrence probability of the event at each time and each location, wherein the parameters are learned on the basis of history information relating to the event, the history information including a time, a location, and an event type, and features of an area in which the location exists, so that a likelihood expressing a combined effect of the event type and the features of the area on the event is optimized.

A third aspect of the present disclosure is a learning method in which a computer executes processing including learning parameters for determining an occurrence probability of an event at each time and each location on the basis of history information relating to the event, the history information including a time, a location, and an event type, and features of an area corresponding to the location, so that a likelihood expressing a combined effect of the event type and the features of the area on the event is optimized.

A fourth aspect of the present disclosure is a prediction method in which a computer executes processing including receiving a predicted time and a predicted location, and predicting the occurrence of an event at the predicted time and the predicted location on the basis of pre-learned parameters for determining an occurrence probability of the event at each time and each location, wherein the parameters are learned on the basis of history information relating to the event, the history information including a time, a location, and an event type, and features of an area in which the location exists, so that a likelihood expressing a combined effect of the event type and the features of the area on the event is optimized.

A fifth aspect of the present disclosure is a program for causing a computer to execute the processing of the learning device described in the first aspect or the prediction device described in the second aspect.

### Effects of the Invention

According to the technology in the disclosure, features of an area can be ascertained, whereby the occurrence of an event can be predicted with a high degree of precision.

### BRIEF DESCRIPTION OF DRAWINGS

FIG. 1 is a block diagram showing a configuration of a learning device according to a first embodiment.

FIG. 2 is a block diagram showing hardware configurations of the learning device and a prediction device.

FIG. 3 is a view showing an example of history information stored in an event history storage device.

FIG. 4 is a view showing an example of area features serving as external information stored in an external information storage device.

FIG. 5 is a flowchart showing a flow of learning processing executed by the learning device.

FIG. 6 is a block diagram showing a configuration of a prediction device according to a second embodiment.

FIG. 7 is a flowchart showing a flow of prediction processing executed by the prediction device.

### DESCRIPTION OF EMBODIMENTS

Example embodiments of the technology in the disclosure will be described below with reference to the figures. Note that in the figures, identical or equivalent constituent elements and parts have been allocated identical reference symbols. Further, dimension ratios in the figures have been exaggerated to facilitate the description and may therefore differ from the actual ratios.

First, the background to and a summary of the present disclosure will be described.

Predicting events such as conflicts caused by armed assaults, terrorism, or gang warfare and disasters such as earthquakes and outbreaks of infectious diseases plays an extremely important role in keeping the general public safe and healthy. For example, if attacks and terrorism by armed groups can be predicted, advance measures such as calling on the general public to evacuate can be taken. If an outbreak of an infectious disease can be predicted, the spread of infections can be forestalled by promoting vaccination.

As noted above, a self-exciting spatio-temporal point process known as the Hawkes process is widely used to predict such events (see NPL 1 and NPL 2). In the Hawkes process, an “intensity function” representing the occurrence probability of the event is assumed to have a self-exciting property. In other words, in the Hawkes process, a phenomenon whereby, when an event occurs, the occurrence probability of an event of the same type increases, or in other words, the value of the intensity function jumps, is modeled. The Hawkes process captures a phenomenon whereby a certain event triggers another event, for example when a large earthquake triggers an earthquake in the surrounding area, or a conflict started by a gang against an enemy organization leads to a retaliatory conflict.

The magnitude of the effect of the event is expressed by parameters of the intensity function. The parameters of the intensity function are normally estimated from data using the maximum likelihood method or the like. The magnitude of the effect of the event is believed to vary according to the event type and external factors. Event types and external factors will be described using a conflict between nations and an outbreak of an infectious disease as examples.

First, an example of a conflict between nations will be described. A case in which the military of a certain country A launches an attack on the military of a country B (corresponding to the event type; described hereafter as event 1-1) will be considered. In such a case, the military of country B may attack the military of country A in retaliation (a phenomenon whereby event 1-1 triggers another event 1-2). The probability of the military of country B launching a retaliatory attack (corresponding to the value of the intensity function) varies according to the type of the initial event, and also varies according to external factors. For example, the event type “the military of a certain country A launches an attack on the military of a country B” may have external factors such as “many casualties” and “no casualties”. In the case of a large-scale attack resulting in many casualties, for example, a retaliatory action (corresponding to the effect of the event) is more likely to occur. Further, the phenomenon whereby event 1-1 triggers another event 1-2 also depends on another external factor, namely the geographical features of the location of the retaliatory attack (corresponding to an external factor). For example, when the military of country B retaliates against an attack by the military of country A, the territory of country A may be targeted. In other words, the magnitude of the effect of each event is determined by a mutual relationship between the event type and external factors such as the existence or the number of casualties and the geographical features of the predicted area. Note that the former is an external factor relating to the event, while the latter is an external factor relating to the features of the area.

Next, an example of an outbreak of an infectious disease will be described. It is assumed that a patient with an infectious disease has been found in a certain location (corresponding to the event type; described hereafter as event 2-1). The way in which a disease is transmitted depends not only on the type of disease but also external factors. In this case, the external factors include, for example, the type of infectious disease, such as “influenza” or “malaria”, the climate, the vaccination rate, the hygiene environment, and so on. Influenza, for example, spreads more easily in seasons with low air temperatures and in countries and regions where vaccination is not common. Malaria, on the other hand, spreads easily in tropical or subtropical regions where mosquitoes, which are the carriers of malaria, live. In order to appropriately model the magnitude of the effect of the event (corresponding to the value of the intensity function) in relation to the event type, i.e., an outbreak of an infectious disease, it is necessary to take external factors such as the type of infectious disease, time-related external information such as weather, and space-related external information such as the extent of vaccination in each country into consideration and learn the mutual relationship therebetween.

As described above, to predict an event with a high degree of precision, it is essential to make effective use of information relating to the event type and external factors. In existing spatio-temporal Hawkes processes, however, this information cannot be taken into consideration.

A method according to this embodiment relates to a technique for predicting a future event on the basis of history information about the occurrence of an event in space-time and external information that affects the occurrence probability of the event. Here, the event is a history of urban conflict, terrorism, gang warfare, or the like, or a record of earthquakes and outbreaks of infectious diseases, for example, and these events will be described below as examples. However, the applicable scope of the method of this embodiment is not limited thereto. The history information expresses the time at which the event occurred, the latitude and longitude of the location where the event occurred, and additional information. Here, the additional information is information appended to each individual event. For example, when a history of terrorism is used as an example, the additional information includes a description of the attacker organization, the target of the attack, and the damage caused thereby, and so on.

Configurations of this embodiment will be described below. A learning device will be described in a first embodiment, and a prediction device will be described in a second embodiment.

### Configuration of Learning Device of First Embodiment

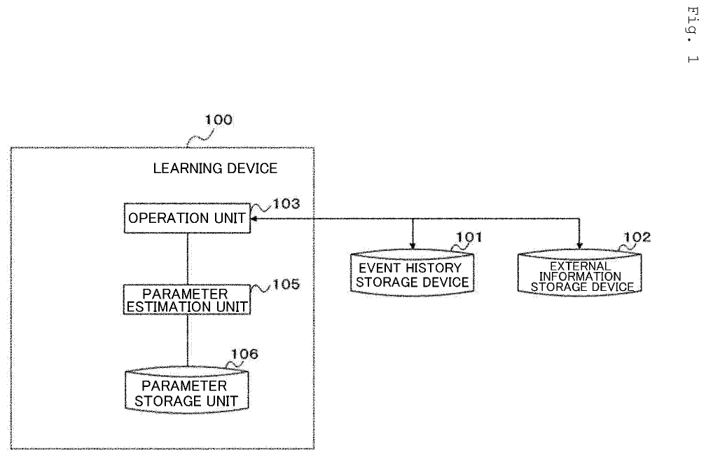

FIG. 1 is a block diagram showing a configuration of a learning device according to a first embodiment.

As shown in FIG. 1, a learning device 100 is connected to an event history storage device 101 and an external information storage device 102 by a network (not shown). The learning device 100 is configured to include an operation unit 103, a parameter estimation unit 105, and a parameter storage unit 106.

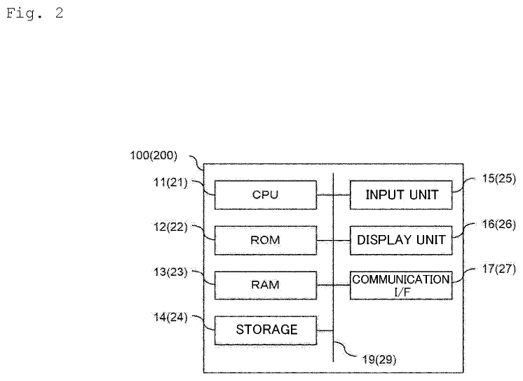

FIG. 2 is a block diagram showing a hardware configuration of the learning device 100.

As shown in FIG. 2, the learning device 100 includes a CPU (Central Processing Unit) 11, a ROM (Read Only Memory) 12, a RAM (Random Access Memory) 13, storage 14, an input unit 15, a display unit 16, and a communication interface (I/F) 17. The respective configurations are connected to each other communicably by a bus 19.

The CPU 11 is a central calculation processing unit that executes various programs and controls the respective units. More specifically, the CPU 11 reads a program from the ROM 12 or the storage 14 and executes the program using the RAM 13 as a working area. The CPU 11 controls the respective configurations described above and performs various types of calculation processing in accordance with the program stored in the ROM 12 or the storage 14. In this embodiment, a learning program is stored in the ROM 12 or the storage 14.

The ROM 12 stores various programs and various data. The RAM 13 stores a program or data temporarily as a working area. The storage 14 is constituted by an HDD (Hard Disk Drive) or an SSD (Solid State Drive) and stores various programs, including an operating system, and various data.

The input unit 15 includes a pointing device such as a mouse and a keyboard, and is used to input various types of input.

The display unit 16 is a liquid crystal display, for example, and displays various information. By employing a touch panel system, the display unit 16 may also function as the input unit 15.

The communication interface 17 is an interface for communicating with another device, such as a terminal, and uses a standard such as Ethernet (registered trademark), FDDI, or Wi-Fi (registered trademark), for example.

The above constitutes the hardware configuration of the learning device 100.

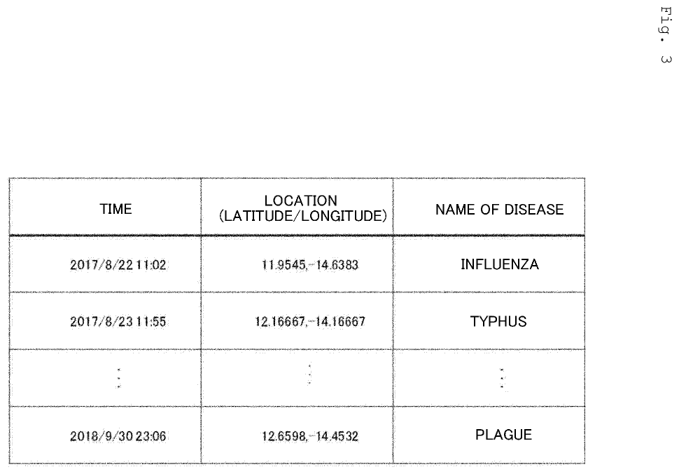

The event history storage device 101 stores history information relating to a spatio-temporal event, which is used during learning processing performed by the learning device 100. In response to a request from the learning device 100, the event history storage device 101 reads the history information relating to the spatio-temporal event and transmits the history information to the learning device 100. The history information is event-related information including a time, a location, and an event type. A conflict between nations, gang warfare, an outbreak of an infectious disease, and so on may be cited as examples of event types. External factors relating to the event are appended to the event type so as to be included in the history information. Here, event-related external factors are information other than the event type, or in other words event-related information relating to the time, location, and event type. The history information is defined as a combination of a time tᵢ ∈T, a latitude and a longitude sᵢ∈S serving as a location, and additional information zᵢ expressing the event type. Here, T×S is a subset of R×R² (where R is an outlined character representing a set of real numbers). Here, the additional information zᵢ is a feature amount appended to each event. In the case of a conflict or gang warfare, the additional information zᵢ represents the attacker, the target of the attack, or the number of casualties. In the case of an infectious disease, the additional information zᵢ represents the type of the infectious disease or a description of symptoms. In this embodiment, a case in which n spatio-temporal events occur up to a time T such that a dataset D={(tᵢ,sᵢ,zᵢ)}^{l}ᵢ₌₁ of data constituted by l={1, . . . , n} is given as the history information will be considered. The event history storage device 101 is constituted by a web server that hosts a website, a database server having a database, or the like. FIG. 3 is a view showing an example of the history information stored in the event history storage device 101.

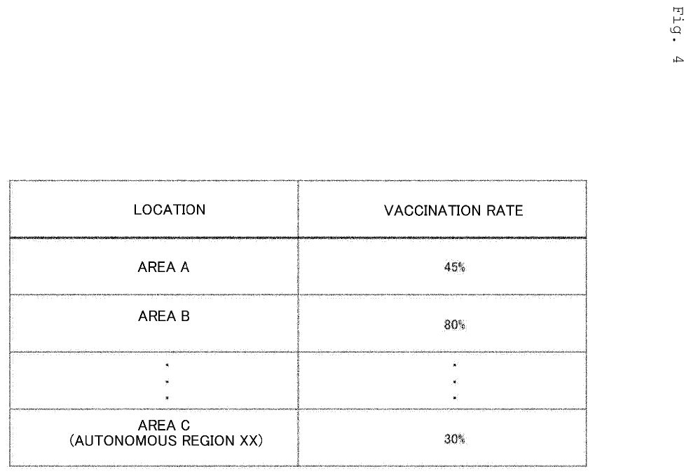

The external information storage device 102 stores external information used in the learning processing performed by the learning device 100. In response to a request from the learning device 100, the external information storage device 102 reads the external information and transmits the external information to the learning device 100. In this embodiment, a case in which external information a representing an area R E S defined in a geographical space S and geographical features within a time interval H E T defined within T is given together with the history information of 1 events is envisaged. The external information a includes, for example, the economic standard and medical standard of each country or each area, as well as transitions therein over time. In other words, the external information a is constituted by features of the area corresponding to the location relating to the event, and serves as an example of external factors relating to the features of the area. In this embodiment, for simplicity, a case in which only the external information a associated with the area is given will be considered. Hereafter, the external information will also be described as the features a of the area. In the case of an infectious disease, the features a of the area express the vaccination implementation rate of the area R, the weather (the air temperature, humidity, and so on) during the time interval H, and so on. Note, however, that the following description can easily be generalized to a case in which external information associated with the time interval is given. Divisions (countries or regions) of the area in the geographical space are represented by R={R₁, R₂, . . . }. The features a of the area are represented by a series {Rᵥ, aᵥ} (Rᵥ∈R) of pairs of an area and a value. y(t, s) is introduced as a function representing external information associated with a time t and a location s. In other words, y(t, s) is a function that returns features aᵥ of an area s∈Rᵥ. The external information storage device 102 is constituted by a web server that hosts a website, a database server having a database, or the like. FIG. 4 is a view showing an example of area features serving as the external information stored in the external information storage device 102. Note that when temporal features are also taken into account, the features of the area are expressed as features a_{u, v} of the area.

Next, respective functional configurations of the learning device 100 will be described. The functional configurations are realized by having the CPU 11 read the learning program stored in the ROM 12 or the storage 14, expand the program to the RAM 13, and execute the program.

The operation unit 103 receives various operations relating to history information D stored in the event history storage device 101 and the area features a stored in the external information storage device 102 as input, and outputs the operations. The various operations include operations for registering, correcting, acquiring, and deleting the stored information, and so on. The operation unit 103 may employ any input means, such as a keyboard, a mouse, a menu screen, a touch panel, and so on. The operation unit 103 may be realized by a device driver of the input means such as a mouse, or control software of a menu screen. In this embodiment, the operation unit 103 acquires and outputs the history information D stored in the event history storage device 101 and the area features a stored in the external information storage device 102 for the purpose of the learning processing in response to input of the various operations.

The parameter estimation unit 105 receives the history information D and the area features a acquired by the operation unit 103 as input, and outputs learned parameters. The parameter estimation unit 105 learns the parameters on the basis of the received history information D and area features a so that a likelihood, which expresses the combined effect of the event type and the features of the area on the event, is optimized. The parameters are parameters for determining the occurrence probability of an event at each time and each location. Specific principles of the parameter estimation performed during the processing for learning the parameters will be described below.

In the parameter estimation of this embodiment, an event triggered by a past event is modeled using a point process. First, an intensity function is designed in accordance with the procedures of a typical point process model. The intensity function is a function expressing the event occurrence probability per unit time. An example of the intensity function is shown below.

An intensity function λ(t, s) for determining the occurrence probability of an event at a time t and a location s is introduced. The frequency of the event varies according to the magnitude of the effect of past events.

$$[ Formula 1 ]$$

$$λ ( t , s ) = μ + {\sum }_{{t}_{j} < t} {ω}_{j} g ( t - {t}_{j} , s - {s}_{j} ) \qquad ( 1 )$$

Here, μ is the event occurrence probability irrespective of the effect of past events. In this case, for simplicity, μ is set at μ=0. Note, however, that the following description can easily be generalized to cases other than μ=0. g is a function known as a trigger function, which is a function for determining the form of self-excitation on the point process model. A trigger function is typically non-negative, and a function such as a kernel function or an exponential decay function is generally used. Here, tⱼ<t represents j^{th} data acquired prior to the time t, within the data of the history information D. Further, to simplify the estimation, a function decomposed into a time term and a space term, as shown below in formula (2), is often used as the trigger function.

[Formula 2]

g(t−tⱼ,s−sⱼ)=h(t−tⱼ)k(s−sⱼ). (2)

Thus, the trigger function is represented by a parameter relating to a time and a parameter relating to a time. In other words, the time-related parameter h (·) is determined by the difference between the time t and the time t prior to the time t, while the time-related parameter k (·) is determined by the difference between the location s corresponding to the time t and a location sᵢ of the data j prior to the time t.

wⱼ is a parameter representing the magnitude of the effect of the j^{th} event in the intensity function. In this embodiment, the magnitude of the effect of each event and the features (in this embodiment, the geographical features) of the subject area are taken into consideration, and therefore, as shown below in formula (3), wⱼ in formula (1) is replaced with the inner product sum of the outputs of two nonlinear functions having these elements as input.

[Formula 3]

wⱼ=Ψ(zⱼ)^{T}Φ(y(t,s)) (3)

Here, Ψ(·), ϕ(·) is an arbitrary nonlinear function having a vector of a length K as output, and a neural network or the like, for example, is used as this function. The formulation described above is based on the assumption that the occurrence probability of an event at the time t and the location s is determined by the combined effect of the type z of a past event and the geographical features y(t, s) of the location s. Hence, the parameter wⱼ representing the magnitude of the effect of each event is represented by a parameter Ψ(·) relating to the event type and a parameter ϕ(·) relating to the features of the area, these parameters replacing w₁. On the basis of the above, a likelihood L of the point process model of this embodiment can be written down as shown below in formula (4).

$$[ Formula 4 ]$$

$$ℒ = {\sum }_{i = 1}^{I} ( log {\sum }_{j : {t}_{i} < t} {Φ ( {z}_{j} )}^{⊤} Ψ ( y ( {t}_{i} , {s}_{i} ) ) h ( {t}_{i} - {t}_{j} ) k ( {s}_{i} - {s}_{j} ) - {\underbrace{{\int }_{{t}_{i}}^{T} {\int }_{S} {Φ ( {z}_{i} )}^{⊤} Ψ ( y ( t , s ) ) h ( t - {t}_{i} ) k ( s - {s}_{i} ) dtds}}_{{Λ}_{i}} ) . \qquad ( 4 )$$

Here, the integral of the second term on the right side is defined as Λᵢ. Λᵢ can be rewritten as shown below in formula (5)

$$[ Formula 5 ]$$

$${Λ}_{i} = {Φ ( {z}_{i} )}^{⊤} ( {\sum }_{{R}_{v} ∈ ℛ} Ψ ( {a}_{v} ) {\int }_{{t}_{i}}^{T} h ( t - {t}_{i} ) dt {\int }_{{R}_{v}} k ( s - {s}_{i} ) ds ) , \qquad ( 5 )$$

Analytical solutions or approximate solutions can be acquired for a large number of trigger functions h(·), k(·) from the integral included in the above formula. During learning, a set of the parameters of Ψ(·), ϕ(·). Hand the parameters of the trigger function h(·), k(·) with which to minimize the likelihood L is estimated. Any method may be used to optimize the parameters. The likelihood L in the above formula can be differentiated for all of the parameters and can therefore be optimized using a gradient method, for example. A backpropagation method can be applied as is likewise when a neural network is assumed as Ψ, ϕ.

As described above, the likelihood L in formula (4) is expressed so as to include the parameter Ψ(·) relating to the event type, the parameter ϕ(·) relating to the features of the area, the parameter h(·) relating to the time, and the parameter k(·) relating to the location. The parameter estimation unit 105 optimizes the parameter Ψ(·) relating to the event type, the parameter ϕ(·) relating to the features of the area, the parameter h(·) relating to the time, and the parameter k(·) relating to the location as the parameters. The parameter estimation unit 105 then stores the parameters for determining the occurrence probability of the event at each time and each location, the parameters having been learned so that the likelihood of formula (4) is optimized, in the parameter storage unit 106.

The parameter storage unit 106 stores a set of the parameters learned by the parameter estimation unit 105. The parameter storage unit 106 may have any configuration as long as the set of optimized parameters can be stored therein and restored thereby. For example, the parameters are stored in a specific area of a database, a pre-installed memory serving as a general-purpose storage device, a hard disk, or the like.

### Actions of Learning Device of First Embodiment

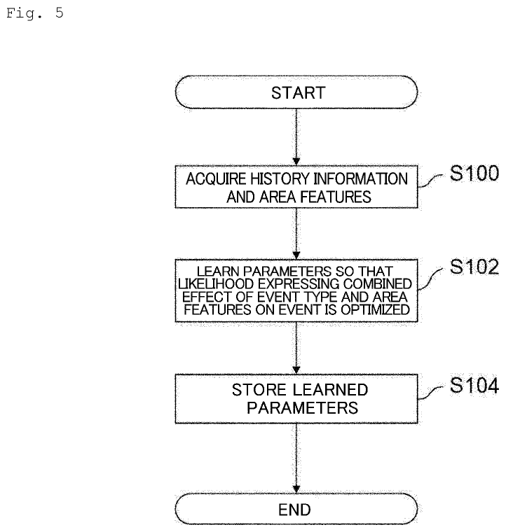

Next, actions of the learning device 100 will be described. FIG. 5 is a flowchart showing a flow of the learning processing performed by the learning device 100. The learning processing is performed by having the CPU 11 read the learning program from the ROM 12 or the storage 14, expand the program to the RAM 13, and execute the program.

In step S100, the CPU 11, acting as the operation unit 103, acquires the history information D stored in the event history storage device 101 and the features a of the area, stored in the external information storage device 102, for the purpose of the learning processing.

In step S102, the CPU 11 learns the parameters on the basis of the history information D and the features a of the area which were acquired in step S100, so that the likelihood expressing the combined effect of the event type and the features of the area on the event is optimized. The parameters are parameters for determining the occurrence probability of the event at each time and each location. In this step, the parameter Ψ(·) relating to the event type, the parameter ϕ(·) relating to the features of the area, the parameter h(·) relating to the time, and the parameter k(·) relating to the location are optimized as the parameters in relation to the likelihood L of formula (4). Note that the processing of step S102 is executed by the CPU 11 acting as the parameter estimation unit 105.

In step S104, the CPU 11, acting as the parameter estimation unit 105, stores the parameters learned in step S102 in the parameter storage unit 106.

With the learning device 100 according to this embodiment, as described above, the features of the area can be ascertained, and as a result, parameters for predicting the occurrence of the event can be learned with a high degree of precision.

### Configuration of Prediction Device of Second Embodiment

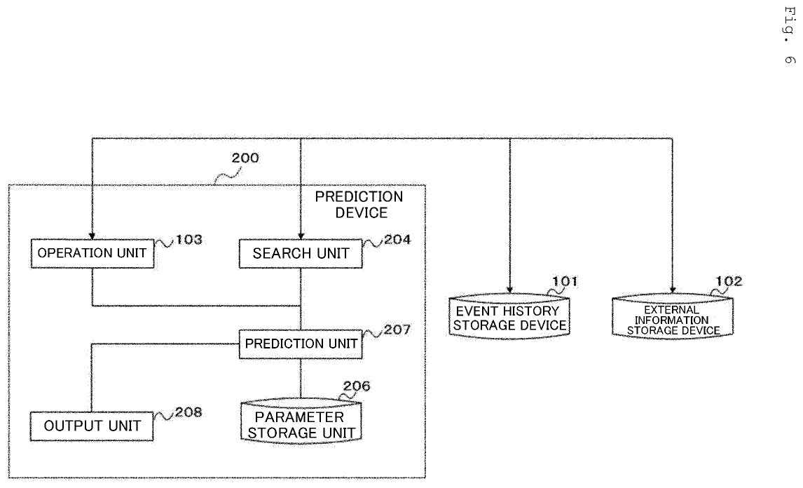

FIG. 6 is a block diagram showing a configuration of a prediction device according to a second embodiment. Note that similar locations to the first embodiment have been allocated identical reference numerals, and description thereof has been omitted.

As shown in FIG. 6, a prediction device 200 is connected to the event history storage device 101 and the external information storage device 102 by a network (not shown). The prediction device 200 is configured to include the operation unit 103, a search unit 204, a parameter storage unit 206, a prediction unit 207, and an output unit 208.

Note that the prediction device 200 may be formed from a similar hardware configuration to the learning device 100. As shown in FIG. 2, the prediction device 200 includes a CPU 21, a ROM 22, a RAM 23, storage 24, an input unit 25, a display unit 26, and a communication I/F 27. The respective configurations are connected to each other communicably by a bus 29. A prediction program is stored in the ROM 22 or the storage 24.

Next, respective functional configurations of the prediction device 200 will be described. The functional configurations are realized by having the CPU 21 read the prediction program stored in the ROM 22 or the storage 24, expand the program to the RAM 23, and execute the program.

The search unit 204 receives a predicted time and a predicted location as input, and outputs the received time and location. The search unit 204 may employ any input means, such as a keyboard, a mouse, a menu screen, a touch panel, or the like. The search unit 204 can be realized by a device driver of the input means such as a mouse, or control software of a menu screen.

Further, having received the input described above, the search unit 204 acquires, from the event history storage device 101 and the external information storage device 102, history information D′ and area features a′ corresponding to the predicted time and location, the history information D′ and area features a′ being required in the prediction processing performed by the prediction unit 207, and then outputs the acquired history information D′ and area features a′.

The parameters for determining the occurrence probability of the event at each time and each location, which have been learned by the learning device 100, are stored in the parameter storage unit 206. In the learning device 100, the parameters are learned on the basis of the history information D relating to the event, which includes the time, the location, and the event type, and the features a of the area in which the location exists. The parameters are learned so that the likelihood of formula (4), which expresses the combined effect of the event type and the features of the area on the event, is optimized. The likelihood L in formula (4) is expressed so as to include the parameter Ψ(·) relating to the event type, the parameter ϕ(·) relating to the features of the area, the parameter h(·) relating to the time, and the parameter k(·) relating to the location. The parameter IF (·) relating to the event type, the parameter ϕ(·) relating to the features of the area, the parameter h(·) relating to the time, and the parameter k(·) relating to the location are optimized as the parameters.

The prediction unit 207 receives, as input, the predicted time and location received by the search unit 204 and the history information D′ and area features a′ acquired by the search unit 204, and outputs a prediction result of the occurrence of the event at the predicted time and location. The prediction unit 207 predicts the occurrence of the event at the predicted time and location received by the search unit 204 on the basis of the history information D′ and area features a′ acquired by the search unit 204 and the parameters stored in the parameter storage unit 206. Here, a plurality of methods for simulating a point process exist, but the method described in reference document 1, which is known as “thinning”, for example, can be applied.
[Reference document 1] OGATA, Yosihiko. On Lewis' simulation method for point processes. IEEE Transactions on Information Theory, Jan. 27, 1981: 23-31.

Here, a specific example of the prediction processing performed by the prediction unit 207 will be described. In prediction processing using a point process model, the search unit 204 receives a predicted time Wₜ and a predicted location Wₛ as input. Wₜ is set as Wₜ=[Tₚ, T_{q}], and is expressed by specifying a start point Tₚ and an endpoint T_{q}. Wₛ is set as Wₛ∈S (where S is an outline character representing a set of real numbers), and is expressed by specifying a subject area Wₛ within the entire area S. Further, the search unit 204 acquires the additional information zᵢ expressing the event type in the history information D′ corresponding to the predicted time Wₜ and location Wₛ. Furthermore, the search unit 204 acquires area features a_{u,v} (=a′) corresponding to the predicted time Wₜ and location Wₛ from the external information storage device 102. The prediction unit 207 acquires the parameter (·) relating to the event type, the parameter ϕ(·) relating to the features of the area, the parameter h(·) relating to the time, and the parameter k(·) relating to the location from the parameter storage unit 206 as the parameters. The prediction unit 207 then executes a simulation shown below in formula (6) in relation to the received predicted time Wₜ and location Wₛ using the acquired parameters, zᵢ, and a_{u, v} in order to predict the occurrence probability of the event.

$$[ Formula 6 ]$$

$$\begin{matrix}𝒩 ( {W}_{T} \times {W}_{S} ) = {\int }_{{W}_{T}} {\int }_{{W}_{S}} λ ( t , s ) dtds \\ = {\sum }_{\text{?}} {Φ ( {x}_{i} )}^{⊤} ( \begin{matrix}{\sum }_{\text{?}} {\sum }_{\text{?}} Ψ ( \text{?} {\int }_{{T}_{p}}^{{T}_{q}} \text{?} [ I ∈ {H}_{\text{?}} ] h ( t - {t}_{i} ) di \\ {\int }_{{W}_{S}} \text{?} [ s ∈ {R}_{v} ] k ( s - {s}_{i} ) ds\end{matrix} ) .\end{matrix} \qquad ( 6 )$$

$$\text{?} \text{indicates text missing or illegible when filed}$$

The output unit 208 receives, as input, the prediction result of the occurrence probability of the event at the predicted time Wₜ and the predicted location Wₛ, as predicted by the prediction unit 207, and outputs the prediction result to the outside. Here, output to the outside is a concept including display on a display, printing using a printer, audio output, transmission to an external device, and so on. The output unit 208 may include an output device such as a display or a speaker. The output unit 208 can be realized by driver software of an output device, driver software of an output device as well as the output device, or the like.

### Actions of Prediction Device of Second Embodiment

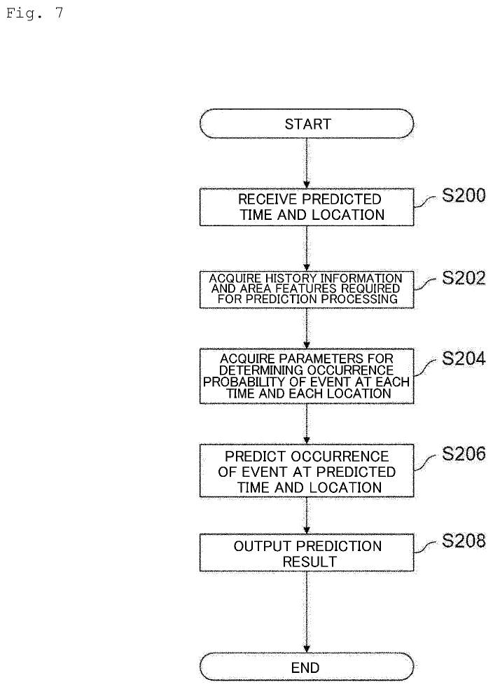

Next, actions of the prediction device 200 will be described. FIG. 7 is a flowchart showing a flow of the prediction processing performed by the prediction device 200. The prediction processing is performed by having the CPU 21 read the prediction program from the ROM 22 or the storage 24, expand the program to the RAM 23, and execute the program.

In step S200, the CPU 21, acting as the search unit 204, receives the predicted time and location.

In step S202, the CPU 21, acting as the search unit 204, acquires, from the event history storage device 101 and the external information storage device 102, the history information D′ and the area features a′ corresponding to the predicted time and location, the history information D′ and area features a′ being required in the prediction processing performed by the prediction unit 207.

In step S204, the CPU 21, acting as the prediction unit 207, acquires the parameters for determining the occurrence probability of the event at each time and each location from the parameter storage unit 206. The acquired parameters are the parameter Ψ(·) relating to the event type, the parameter ϕ(·) relating to the features of the area, the parameter h(·) relating to the time, and the parameter k(·) relating to the location.

In step S206, the CPU 21 predicts the occurrence of the event at the predicted time and location received in step S200 on the basis of the history information D′ and area features a′ acquired in step S202 and the parameters acquired in step S204. The occurrence of the event is predicted as the occurrence probability of the event at the predicted time and location. Note that the processing of step S206 is executed by the CPU 21 acting as the prediction unit 207.

In step S208, the CPU 21, acting as the output unit 208, outputs the occurrence probability of the event at the predicted time and location, predicted in step S206, to the outside as a prediction result.

With the prediction device 200 according to this embodiment, as described above, the features of the area can be ascertained, and as a result, the occurrence of the event can be predicted with a high degree of precision.

### Experimental Example

An experimental example of the learning processing performed by the learning device 100 of the first embodiment and the prediction processing performed by the prediction device 200 of the second embodiment will now be illustrated. Here, three datasets, namely a history of armed conflict (Armed Conflict), a history of terrorism (Terrorism), and an outbreak history of a disease (Disease), were used as event data.

An example of the calculations performed in the method proposed by this embodiment will be illustrated. In this experiment, event data observed over a test period were used. The event data are a dataset D of data x including features of environments of respective events X*={xₗ₊₁, . . . , x_{l+Nt}} (the subscript Nt being Nₜ), and are constituted by data observed over a test period [T, T+ΔT]. The parameters of the likelihood were optimized by inserting the event data observed over the test period into formula (7), shown below.

$$[ Formula 7 ]$$

$${ℒ}^{*} = {\sum }_{i = l + 1}^{I + {N}_{i}} ( log {\sum }_{j : {t}_{i} < t} {Φ ( {z}_{j} )}^{⊤} Ψ ( y ( {t}_{i} , {s}_{i} ) ) h ( {t}_{i} - {t}_{j} ) k ( {s}_{i} - {s}_{j} ) - {\int }_{{t}_{i}}^{T} {\int }_{S} {Φ ( {z}_{i} )}^{⊤} Ψ ( y ( t , s ) ) h ( t - {t}_{i} ) k ( s - {s}_{i} ) dtds ) . \qquad ( 7 )$$

A comparison with three existing methods (HP, Hawkes, NPP) was performed using the likelihood (the test likelihood) relating to the event data observed over the test period. The values shown below on table 1 are test likelihoods, and higher values indicate a superior prediction performance.

TABLE 1

| | Armed Conflict | Terrorism | Disease |
|---|---|---|---|
| HPP | 6.910 | 7.544 | 7.741 |
| HAWKES | 6.980 | 7.225 | 7.104 |
| NPP | 7.082 | 7.356 | 7.195 |
| Method Proposed | 9.312 | 9.704 | 10.076 |

The three existing methods can be summarized as follows. (1) HPP (Spatio-temporal homogeneous Poisson Process): a simple point process model in which a fixed intensity is assumed regardless of the time and location. (2) Hawkes (Spatio-temporal Hawkes Process) (see NPL 1): the intensity of this model is described in formula (1). Neither additional information nor external information is taken into account. An identical function to that of the method proposed by this embodiment was used as the trigger function. (3) NPP (Spatio-temporal Hawkes Process with event features): a simple expansion of the Hawkes model, in which only the additional information zᵢ expressing the event type is taken as the input of the intensity λ(t, s). This corresponds to a model in which ϕ(·) is deleted from formula (3) and K is fixed at K=1.

According to table 1, the method proposed by the present disclosure gives a superior prediction performance to those of all of the existing methods of (1) to (3).

Note that in the embodiments described above, the learning processing or the prediction processing that is executed by the CPU by reading software (a program) may be executed by various processors other than a CPU. A PLD (Programmable Logic Device) such as an FPGA (Field-Programmable Gate Array), the circuit configuration of which can be modified post-manufacture, a dedicated electrical circuit serving as a processor having a circuit configuration specially designed to execute specific processing, such as an ASIC (Application Specific Integrated Circuit), and so on may be cited as examples of the processor in this case. Further, the learning processing or the prediction processing may be executed by one of these various processors or by a combination of two or more processors of the same type or different types (for example, a plurality of FPGAs, a combination of a CPU and an FPGA, and so on). Furthermore, more specifically, the hardware structure of these various processors is an electrical circuit combining circuit elements such as semiconductor elements.

Moreover, in the embodiments described above, an aspect in which the learning program is stored (installed) in advance in the storage 14 was described, but the present disclosure is not limited thereto. The program may be provided by being stored on a non-transitory storage medium such as a CD-ROM (Compact Disk Read Only Memory), a DVD-ROM (Digital Versatile Disk Read Only Memory), or a USB (Universal Serial Bus) memory. In addition, the program may be downloaded from an external device over a network. These points relating to the learning program apply similarly to the prediction program.

The following additional remarks are disclosed in relation to the embodiments described above.

(Additional Remark 1)

A learning device including:

a memory; and

at least one processor connected to the memory, wherein

the processor is configured to learn parameters for determining an occurrence probability of an event at each time and each location on the basis of history information relating to the event, the history information including a time, a location, and an event type, and features of an area corresponding to the location, so that a likelihood expressing a combined effect of the event type and the features of the area on the event is optimized.

(Additional Remark 2)

A non-transitory storage medium storing a learning program for causing a computer to execute learning of parameters for determining an occurrence probability of an event at each time and each location on the basis of history information relating to the event, the history information including a time, a location, and an event type, and features of an area corresponding to the location, so that a likelihood expressing a combined effect of the event type and the features of the area on the event is optimized.

### REFERENCE SIGNS LIST

100 Learning device
101 Event history storage device
102 External information storage device
103 Operation unit
105 Parameter estimation unit
106 Parameter storage unit
200 Prediction device
204 Search unit
206 Parameter storage unit
207 Prediction unit
208 Output unit

1. A learning device comprising circuitry configured to execute a method comprising:
learning parameters for determining an occurrence probability of an event at each time and each location based on history information associated with the event, the history information including a time, a location, and an event type, and features of an area corresponding to the location, so that a likelihood expressing a combined effect of the event type and the features of the area on the event is optimized.

2. The learning device according to claim 1, wherein
the likelihood so as to includes a parameter corresponding to the event type and a parameter corresponding to the features of the area, the respective parameters replacing a parameter expressing the magnitude of the effect of each event in an intensity function used to determine the occurrence probability of the event at each time and each location, and
the circuitry further configured to executed a method comprising:
optimizing the parameter relating to the event type and the parameter corresponding to the features of the area as the parameters.

3. The learning device according to claim 2, wherein
the likelihood includes a parameter relating to the time and a parameter relating to the location, and
the circuitry further configured to executed a method comprising:
optimizing the parameter corresponding to the event type, the parameter corresponding to the features of the area, the parameter corresponding to the time, and the parameter corresponding to the location as the parameters.

4. A prediction device comprising circuitry configured to execute a method comprising:
receiving a predicted time and a predicted location; and
predicting the occurrence of an event at the predicted time and the predicted location based on pre-learned parameters for determining an occurrence probability of the event at each time and each location, wherein
the parameters are learned based on history information associated with the event, the history information including a time, a location, and an event type, and features of an area in which the location exists, so that a likelihood expressing a combined effect of the event type and the features of the area on the event is optimized.

5. (canceled)

6. A computer-implemented method for predicting, the method comprising:
receiving a predicted time and a predicted location; and
predicting an occurrence of an event at the predicted time and the predicted location based on pre-learned parameters for determining an occurrence probability of the event at each time and each location, wherein
the parameters are learned based on history information associated with the event, the history information including a time, a location, and an event type, and features of an area in which the location exists, so that a likelihood expressing a combined effect of the event type and the features of the area on the event is optimized.

7. (canceled)

8. The learning device according to claim 3, wherein the event type includes a feature amount associated with an attacker of an attack, a target of the attack, or a number of casualties during the attack.

9. The learning device according to claim 3, wherein the event type includes a feature amount associated with a type of an infectious disease or a description of symptoms for the infectious disease.

10. The learning device according to claim 3, where the features of the area include data indicating economic standard or medical standard associated with the location.

11. The learning device according to claim 3, where the features of the area include data indicating vaccination implementation rate of the location or weather at the location.

12. The prediction device according to claim 4, wherein the likelihood includes a parameter corresponding to the event type and a parameter corresponding to the features of the area, the respective parameters replacing a parameter expressing the magnitude of the effect of each event in an intensity function used to determine the occurrence probability of the event at each time and each location, and
the parameters are optimized based on the parameter corresponding to the event type and the parameter corresponding to the features of the area.

13. The computer-implemented method according to claim 6, wherein the likelihood includes a parameter corresponding to the event type and a parameter corresponding to the features of the area, the respective parameters replacing a parameter expressing the magnitude of the effect of each event in an intensity function used to determine the occurrence probability of the event at each time and each location, and
the parameters are optimized based on the parameter corresponding to the event type and the parameter corresponding to the features of the area.

14. The prediction device according to claim 12, wherein
the likelihood includes a parameter relating to the time and a parameter relating to the location,
the parameters are optimized based at least on:
the parameter corresponding to the event type,
the parameter corresponding to the features of the area,
the parameter corresponding to the time, or
the parameter corresponding to the location as the parameters.

15. The computer-implemented method according to claim 13, wherein
the likelihood includes a parameter relating to the time and a parameter relating to the location,
the parameters are optimized based at least on:
the parameter corresponding to the event type,
the parameter corresponding to the features of the area,
the parameter corresponding to the time, or
the parameter corresponding to the location as the parameters.

16. The prediction device according to claim 14, wherein the event type includes a feature amount associated with an attacker of an attack, a target of the attack, or a number of casualties during the attack.

17. The prediction device according to claim 14, wherein the event type includes a feature amount associated with a type of an infectious disease or a description of symptoms for the infectious disease.

18. The prediction device according to claim 14, where the features of the area include data indicating economic standard or medical standard associated with the location.

19. The prediction device according to claim 14, where the features of the area include data indicating vaccination implementation rate of the location or weather at the location.

20. The computer-implemented method according to claim 15, wherein the event type includes a feature amount associated with an attacker of an attack, a target of the attack, or a number of casualties during the attack.

21. The computer-implemented method according to claim 15, wherein the event type includes a feature amount associated with a type of an infectious disease or a description of symptoms for the infectious disease, and
where the features of the area include data indicating vaccination implementation rate of the location or weather at the location.

22. The computer-implemented method according to claim 15, where the features of the area include data indicating vaccination implementation rate of the location or weather at the location.
